(19) United States
(12) Patent Application Publication
(10) Pub. No.: US 2023/0272121 A1
(43) Pub. Date: Aug. 31, 2023

(54) A WOOD-DERIVED CARBOHYDRATE COMPOSITION

(71) Applicant: UPM-KYMMENE CORPORATION, Helsinki (FI)

(72) Inventors: Juha Tamper, Levanen (FI); Jere Salminen, Lappeenranta (FI); Meri Ventola, Leipzig (DE)

(21) Appl. No.: 18/009,574

(22) PCT Filed: Jun. 9, 2021
(86) PCT No.: PCT/FI2021/050431

§ 371 Date: Dec. 9, 2022

(30) Foreign Application Priority Data
Jun. 12, 2020 (FI) 20205614

(51) Int. Cl. C08B 1/00; C13K 1/02; C13K 13/00; C08H 8/00

(52) U.S. Cl. CPC C08B 1/003; C13K 1/02; C13K 13/002; C08H 8/00

(57) ABSTRACT

A wood-derived carbohydrate composition is disclosed. The wood-derived carbohydrate composition comprises monomeric C6 sugars and monomeric C5 sugars in a total amount of at least 80 weight-% based on the total dry matter content of the carbohydrate composition. The ratio of the monomeric C5 sugars to the monomeric C6 sugars is at most 0.15. Disclosed is also a method for producing a wood-derived carbohydrate composition.

### CROSS-REFERENCE TO RELATED APPLICATIONS

This application is a U.S. National Phase Entry of International Application No. PCT/FI2021/050431, filed on Jun. 9, 2021, which claims the benefit of and priority To FI Application No. 20205614, filed Jun. 12, 2020, each of which is hereby incorporated by reference herein in its entirety.

### TECHNICAL FIELD

The present disclosure relates to a wood-derived carbohydrate composition comprising monomeric C6 sugars and monomeric C5 sugars. Further, the present disclosure relates to a method for producing a wood-derived carbohydrate composition.

### BACKGROUND

Different methods are known for converting bio-based raw material, such as lignocellulosic biomass, into a liquid stream of various sugars. Being able to provide sufficiently pure carbohydrate composition with properties suitable for further applications, such a production of mono-ethylene glycol or ethanol, has still remained as a task for researchers.

### SUMMARY

A wood-derived carbohydrate composition is disclosed. The composition may comprise monomeric C6 sugars and monomeric C5 sugars in a total amount of at least 80 weight-% based on the total dry matter content of the carbohydrate composition. The ratio of the monomeric C5 sugars to the monomeric C6 sugars may be at most 0.15.

A method for producing a wood-derived carbohydrate composition is also disclosed. The method may comprise:

i) providing a wood-based feedstock originating from wood-based raw material and comprising wood chips, and subjecting the wood-based feedstock to pretreatment to form a slurry;

ii) separating the slurry into a liquid fraction and a fraction comprising solid cellulose particles by a first solid-liquid separation process to form a fraction comprising solid cellulose particles having a total dry matter content of 15-50 weight-%, wherein the first solid-liquid separation process comprises washing the fraction comprising solid cellulose particles until the amount of soluble organic components in the fraction comprising solid cellulose particles is 0.5-5 weight-% based on the total dry matter content;

iii) optionally, diluting the separated fraction comprising solid cellulose particles to a total dry matter content of 8-20 weight-%;

iv) subjecting the fraction comprising solid cellulose particles to enzymatic hydrolysis to form a hydrolysis product, wherein the fraction comprising solid cellulose particles has a total dry matter content of 8-20 weight-%; and

v) separating the hydrolysis product into a solid fraction comprising lignin and a liquid carbohydrate fraction by a second solid-liquid separation process to recover the liquid carbohydrate fraction as a wood-derived carbohydrate composition.

Further is disclosed a wood-derived carbohydrate composition obtainable by the method as disclosed in the current specification.

### BRIEF DESCRIPTION OF THE DRAWINGS

The accompanying drawing, which is included to provide a further understanding of the embodiments and constitute a part of this specification, illustrates an embodiment. In the drawing:

FIG. 1 presents a flow chart of one embodiment of the method for producing a wood-derived carbohydrate composition.

### DETAILED DESCRIPTION

A wood-derived carbohydrate composition is disclosed. The carbohydrate composition may comprise monomeric C6 sugars and monomeric C5 sugars in a total amount of at least 80 weight-% based on the total dry matter content of the carbohydrate composition, wherein the ratio of the monomeric C5 sugars to the monomeric C6 sugars is at most 0.15.

Further, a method for producing a wood-derived carbohydrate composition is also disclosed. The method may comprise:

i) providing a wood-based feedstock originating from wood-based raw material and comprising wood chips, and subjecting the wood-based feedstock to pretreatment to form a slurry;

ii) separating the slurry into a liquid fraction and a fraction comprising solid cellulose particles by a first solid-liquid separation process to form a fraction comprising solid cellulose particles having a total dry matter content of 15-50 weight-%, wherein the first solid-liquid separation process comprises washing the fraction comprising solid cellulose particles until the amount of soluble organic components in the fraction comprising solid cellulose particles is 0.5-5 weight-% based on the total dry matter content;

iii) optionally, diluting the separated fraction comprising solid cellulose particles to a total dry matter content of 8-20 weight-%;

iv) subjecting the fraction comprising solid cellulose particles to enzymatic hydrolysis to form a hydrolysis product, wherein the fraction comprising solid cellulose particles has a total dry matter content of 8-20 weight-%; and

v) separating the hydrolysis product into a solid fraction comprising lignin and a liquid carbohydrate fraction by a second solid-liquid separation process to recover the liquid carbohydrate fraction as a wood-derived carbohydrate composition.

Further is disclosed a wood-derived carbohydrate composition obtainable by the method as disclosed in the current specification. In one embodiment, the wood-derived carbohydrate composition obtainable by the method as disclosed in the current specification is the wood-derived carbohydrate composition as disclosed in the current specification. I.e. the wood-derived carbohydrate composition disclosed in the current specification may be produced by the method as disclosed in the current specification.

The expression “liquid carbohydrate fraction” may refer to a liquid fraction comprising (soluble) carbohydrates. The liquid carbohydrate fraction may be recovered in the method as disclosed in the current specification as the wood-derived carbohydrate composition.

The wood-derived carbohydrate composition as disclosed in the current specification relates to a composition that comprises carbohydrates but may also in addition comprise additional components and/or elements e.g. as disclosed in the current specification. Thus, the “wood-derived carbohydrate composition” may be considered as a “wood-derived carbohydrate-containing composition” or a “wood-derived composition comprising carbohydrates”.

The expression “total dry matter content” may refer to the total amount of solids including suspended solids and soluble or dissolved solids. The total dry matter content may be determined after removing the liquid from a sample followed by drying at a temperature of 45° C. for 24 hours. The effectiveness of the liquid removal may be assured by weighing the sample, drying for a further two hours at the specified temperature, and reweighing the sample. If the measured weights are essentially the same, the drying has been complete, and the total weight may be recorded.

In one embodiment, the ratio of the monomeric C5 sugars to the monomeric C6 sugars in the carbohydrate composition is at most 0.1, or at most 0.05, or at most 0.03, or at most 0.015. In one embodiment, the ratio of monomeric C5 sugars to the monomeric C6 sugars is 0.015-0.15, or 0.03-0.1, or 0.03-0.05. The inventors surprisingly found out that by the method as disclosed in the current specification, one is able to produce a wood-derived carbohydrate composition comprising a high content of monomeric C6 sugars. By the method as disclosed in the current specification, the C5 sugars may be efficiently removed from the carbohydrate composition. Soluble impurities may also be removed with the C5 sugars.

Separating the liquid fraction and the fraction comprising solid cellulose particles by a first solid-liquid separation process, which comprises washing, in step ii) may reduce the amount of soluble C5 sugars by 80-95 weight-%, or 80-90 weight-%, or 85-90 weight-% from the amount present in the slurry. In one embodiment, the amount of C5 sugars is reduced by at least 80 weight-%, or at least 85 weight-%, or at least 90 weight-%, or at least 95 weight-%, as a result of step ii).

The amount of monomeric C5 sugars, monomeric C6 sugars as well as the amount of oligomeric C5 sugars and oligomeric C6 sugars may be determined both qualitatively and quantitatively by high-performance liquid chromatography (HPLC) by comparing to standard samples. Examples of analysis methods can be found in e.g. Sluiter, A., et al., “Determination of sugars, byproducts, and degradation products in liquid fraction process samples”, Technical Report, National Renewable Energy Laboratory, 2008, and Sluiter, A., et al., “Determination of Structural Carbohydrates and Lignin in Biomass”, Technical Report, National Renewable Energy Laboratory, revised 2012.

As used herein, any weight-percentages are given as percent of the total dry matter content of the carbohydrate composition unless specified otherwise. Similarly, other fractions of weight (ppm etc.) may also denote a fraction of the total dry matter content of the carbohydrate composition unless specified otherwise.

By the expression “C5 sugars” should be understood in this specification, unless otherwise stated, as referring to xylose, arabinose, or any mixture or combination thereof. By the expression “C6 sugars” should be understood in this specification, unless otherwise stated, as referring to glucose, galactose, mannose, fructose, or any mixture or combination thereof. By the expression that the sugar is “monomeric” should be understood in this specification, unless otherwise stated, as referring to a sugar molecule present as a monomer, i.e. not coupled or connected to any other sugar molecule(s).

In the current specification the amounts of different components/elements in the wood-derived carbohydrate composition are presented in weight-% based on the total dry matter content of the carbohydrate composition. In this specification the term “total dry matter content of the carbohydrate composition” may refer to the weight of the carbohydrate composition as determined after removing the liquid from the carbohydrate composition followed by drying the same at a temperature of 45° C. for 24 hours. The effectiveness of the liquid removal may be assured by weighing the sample, drying for a further two hours at the specified temperature, and reweighing the sample. If the measured weights are the same, the drying has been complete, and the total weight may be recorded.

As is clear to the skilled person, the total amount of the different components/elements in the wood-derived carbohydrate composition may not exceed 100 weight-%. The amount in weight-% of the different components/elements in the wood-derived carbohydrate composition may vary within the given ranges.

In one embodiment, the monomeric C5 sugars are xylose and/or arabinose. In one embodiment, the monomeric C6 sugars are glucose, galactose, and/or mannose.

The carbohydrate composition may comprise monomeric C6 sugars and monomeric C5 sugars in a total amount of 80-95 weight-%, or 82-94 weight-%, or 85-93 weight-%, or 90-92 weight-%, based on the total dry matter content of the carbohydrate composition.

In one embodiment, the monomeric C6 sugars are present in an amount of at least 80 weight-%, or at least 85 weight-%, or at least 90 weight-% based on the total dry matter content of the carbohydrate composition. In one embodiment, the monomeric C5 sugars are present in an amount of at most 10 weight-%, or at most 8 weight-%, or at most 6 weight-%, or at most 4 weight-%, or at most 3 weight-% based on the total dry matter content of the carbohydrate composition. In one embodiment, the monomeric C5 sugars are present in an amount of 1-10 weight-%, or 1-8 weight-%, or 1-6 weight-%, or 1-4 weight-%, or 1-3 weight-% based on the total dry matter content of the carbohydrate composition.

The carbohydrate composition may comprise oligomeric C6 sugars and oligomeric C5 sugars in a total amount of 0.5-5 weight-%, or 1-3 weight-%, based on the total dry matter content of the carbohydrate composition. By the expression that the sugar is “oligomeric” should be understood in this specification, unless otherwise stated, as referring to a sugar molecule consisting of two or more monomers coupled or connected to each other.

In one embodiment, the oligomeric C5 sugars are xylose and/or arabinose. In one embodiment, the carbohydrate composition does not comprise oligomeric C5 sugars. In one embodiment, the oligomeric C6 sugars are glucose, galactose, mannose, and/or fructose.

The efficiency of the washing carried out in step ii) may be evaluated by analyzing the liquid carbohydrate fraction to determine its composition quantitatively and/or qualitatively. The analysis may be used to determine e.g. the amounts and types of impurities present in the liquid carbohydrate fraction as well as the absolute and relative amounts of C5 sugars and C6 sugars. Non-limiting examples of such a method for determining the presence of various impurities include, but are not limited to, conductivity, optical purity (e.g. color or turbidity), density of the liquid carbohydrate fraction.

In one embodiment, the efficiency of the washing carried out in step ii) is evaluated by analyzing the fraction comprising solid cellulose particles to determine the quantity of soluble sugars present in the fraction comprising solid cellulose particles. Non-limiting examples of such a method for determining the presence of various impurities include, but are not limited to, conductivity, optical purity (e.g. color or turbidity), density of the liquid carbohydrate fraction.

In one embodiment, the conductivity of a 10% aqueous solution of the carbohydrate composition is 0.5-10 mS/cm, or 0.5-5 mS/cm, or 0.5-2 mS/cm, when determined according to SFS-EN 27888 (1994). The value of the conductivity may be used to determine the efficiency of the washing taking place in step ii). I.e. the value of conductivity may be used to determine the amount of soluble lignin present.

In one embodiment, the ICUMSA color value of an aqueous solution of the carbohydrate composition is at most 20 000 IU, or at most 30 000 IU, or at most 40 000 IU, or at most 50 000 IU, when measured using a modified ICUMSA GS1 method without adjusting the pH of the sample to be analyzed and filtering the sample through a 0.45 μm filter before analysis. In one embodiment, the ICUMSA color value of an aqueous solution of the carbohydrate composition is 10 000-50 000 IU, 15 000-40 000 IU, or 20 000-35 000 IU, when measured using a modified ICUMSA GS1 method without adjusting the pH of the sample to be analyzed and filtering the sample through a 0.45 μm filter before analysis.

The carbohydrate composition may comprise organic and/or inorganic impurities (including soluble lignin) in an amount of at most 20 weight-%, or at most 12 weight-%, or at most 10 weight-%, or at most 8 weight-%, or at most 5 weight-%, or at most 3 weight-%, or at most 2 weight-%, based on the total dry matter content of the carbohydrate composition. The carbohydrate composition may comprise organic and/or inorganic impurities (including lignin) in an amount of 2-20 weight-%, or 3-15 weight-%, or 4-10 weight-%, or 5-8 weight-%, based on the total dry matter content of the carbohydrate composition. The carbohydrate composition may comprise organic impurities in an amount of 1-9 weight-%, or 2-8 weight-%, or 3-7 weight-%, based on the total dry matter content of the carbohydrate composition. The carbohydrate composition may comprise inorganic impurities in an amount of 0.05-2 weight-%, or 0.1-1.5 weight-%, or 0.2-1 weight-%, based on the total dry matter content of the carbohydrate composition.

Organic acids can be mentioned as examples of organic impurities. Non-limiting examples of organic impurities are oxalic acid, citric acid, succinic acid, formic acid, acetic acid, levulinic acid, 2-furoic acid, 5-hydroxymethylfurfural (5-HMF), furfural, glycolaldehyde, glyceraldehyde, as well as various acetates, formiates, and other salts or esters. The quality and quantity of organic impurities in the carbohydrate composition may be determined using e.g. a HPLC coupled with e.g. a suitable detector, infrared (IR) spectroscopy, ultraviolet-visible (UV-VIS) spectroscopy, or nuclear magnetic resonance (NMR) spectrometry. Examples of organic impurities that may be present in the carbohydrate composition are listed in below table 1.

TABLE 1
Organic impurities and their amounts

| | Level at most (ppm or |
|---|---|
| | mg/kg of total dry matter |
| | content of carbohydrate |
| Impurity | composition) |
| Oxalic acid | 50 |
| Citric acid | 2000 |
| Succinic acid | 6000 |
| Formic acid | 3000 |
| Acetic acid | 6000 |
| Levulinic acid | 50 |
| Total carboxylic acids | 18000 |
| 2-Furoic acid | 150 |
| 5-Hydroxymethyl furfural | 200 |
| Furfural | 50 |
| Total furans | 400 |
| Glycolaldehyde | 50 |
| Glyceraldehyde | 200 |

The inorganic impurities may be e.g. a soluble inorganic compound in the form of various salts. The inorganic impurities may be salts of the group of elements consisting of Al, As, B, Ca, Cd, Cl, Co, Cr, Cu, Fe, K, Mg, Mn, Mo, Na, Ni, P, Pb, S, Se, Si, and Zn. The amounts of inorganic impurities in the carbohydrate composition can be analyzed using inductively coupled plasma-optical emission spectroscopy (ICP-OES) according to standard SFS-EN ISO 11885:2009. Examples of organic impurities that may be present in the carbohydrate composition are listed in below table 2.

TABLE 2
Inorganic impurities and their amounts

| | Level at most (ppm or |
|---|---|
| | mg/kg of total dry matter |
| | content of carbohydrate |
| Element | composition) |
| Aluminium, Al | 50 |
| Arsenic, As | 50 |
| Boron, B | 50 |
| Calcium, Ca | 200 |
| Cadmium, Cd | 50 |
| Chloride, Cl | 300 |
| Cobalt, Co | 50 |
| Chromium, Cr | 50 |
| Copper, Cu | 50 |
| Iron, Fe | 50 |
| Potassium, K | 1000 |
| Magnesium, Mg | 100 |
| Manganese, Mn | 50 |
| Molybdenum, Mo | 50 |
| Sodium, Na | 7000 |
| Nickel, Ni | 50 |
| Phosphorus, P | 1500 |
| Lead, Pb | 50 |
| Sulphur, S | 800 |
| Selenium, Se | 50 |
| Silicon, Si | 50 |
| Zinc, Zn | 50 |

In one embodiment, the carbohydrate composition comprises sulphur in an amount of 0.01-0.3 weight-%, or 0.02-0.2 weight-%, or 0.03-0.1 weight-%, based on the total dry matter content of the carbohydrate composition. The amount of sulphur may be determined according to standard SFS-EN ISO 11885 (2009).

The carbohydrate composition may comprise nitrogen in an amount of at most 0.5%, or at most 0.3 weight-%, or at most 0.25 weight-%, or at most 0.2 weight-%, or at most 0.15 weight-%, based on the total dry matter content of the carbohydrate composition when measured as total nitrogen content of the carbohydrate composition. The carbohydrate composition may comprise nitrogen in an amount of 0.01-1.0 weight-%, or 0.03-0.75 weight-%, or 0.05-0.5 weight-%, based on the total dry matter content of the carbohydrate composition when measured as total nitrogen content of the carbohydrate composition. The total amount of nitrogen present in the carbohydrate composition may be determined using any suitable method known to a person skilled in the art, e.g. the Kjeldahl method or catalytic thermal decomposition/chemiluminescence methods.

The carbohydrate composition may comprise soluble lignin in an amount of at most 5 weight-%, or at most 3 weight-%, or at most 1.5 weight-%, or at most 1 weight-%, based on the total dry matter content of the carbohydrate composition. The carbohydrate composition may comprise soluble lignin in an amount of 0.5-5 weight-%, 0.5-3 weight-% or 0.5-2 weight-%, based on the total dry matter content of the carbohydrate composition. The presence of soluble lignin in the carbohydrate composition may evidence that the carbohydrate composition is derived from wood.

The amount of soluble lignin may be determined by UV-VIS absorption spectroscopy in the following manner: The amount of soluble lignin present in the carbohydrate composition is determined by diluting a sample of carbohydrate composition so that its absorbance at 205 nm is 0.2-0.7 AU when compared to a reference sample of pure water and using a cuvette with a path length of 1 cm. The soluble lignin content of the sample in mg/l may then be calculated using the following equation

$$x = ( \frac{A}{a} ) \times D$$

where A is absorbance of the sample, a is the absorptivity coefficient 0.110 l/mgcm, and D is a dilution factor.

The total dry matter content of the wood-derived carbohydrate composition may be 5-15 weight-%, or 6-13 weight-%, or 7-11 weight-% when determined after drying at a temperature of 45° C. for 24 hours.

The method for producing the wood-derived carbohydrate composition may comprise subjecting a wood-based feedstock to pretreatment. By the expression “pretreating” or “pretreatment” should be understood in this specification, unless otherwise stated, (a) process(es) conducted to convert wood-based feedstock to a slurry. The slurry may be separated into a fraction comprising solid cellulose particles and a liquid fraction. The fraction comprising solid cellulose particles may further include an amount of lignocellulose particles as well as lignin particles in free form. Lignocellulose comprises lignin chemically bonded to the cellulose particles.

The wood-based raw material may be selected from a group consisting of hardwood, softwood, and their combination. The wood-based raw material may e.g. originate from pine, poplar, beech, aspen, spruce, eucalyptus, ash, or birch. The wood-based raw material may also be any combination or mixture of these. The wood-based raw material may be broadleaf wood. Preferably the wood-based raw material is broadleaf wood due to its relatively high inherent sugar content, but the use of other kinds of wood is not excluded. The broadleaf wood may be selected from a group consisting of beech, birch, ash, oak, maple, chestnut, willow, poplar, and any combination of mixture thereof.

In one embodiment, the wood-derived carbohydrate composition is a broadleaf-derived carbohydrate composition. The wood-derived carbohydrate composition may thus be produced from wood, such as broadleaf wood, hardwood, softwood, etc.

In general, wood and wood-based raw materials are essentially composed of cellulose, hemicellulose, lignin, and extractives. Cellulose is a polysaccharide consisting of a chain of glucose units. Hemicellulose comprises polysaccharides, such as xylan, mannan, and glucan.

Providing the wood-based feedstock in step i) may comprise subjecting wood-based raw material to a mechanical treatment selected from debarking, chipping, dividing, cutting, beating, grinding, crushing, splitting, screening, and/or washing the wood-based raw material to form the wood-based feedstock.

Thus, providing the wood-based feedstock originating from the wood-based raw material may comprise subjecting the wood-based raw material to a mechanical treatment to form a wood-based feedstock. The mechanical treatment may comprise debarking, chipping, dividing, cutting, beating, grinding, crushing, splitting, screening, and/or washing the wood-based raw material. During the mechanical treatment e.g. wood logs can be debarked and/or wood chips of the specified size and structure can be formed. The formed wood chips can also be washed, e.g. with water, in order to remove e.g. sand, grit, and stone material therefrom. Further, the structure of the wood chips may be loosened before the pretreatment step. The wood-based feedstock may contain a certain amount of bark from the wood logs.

Providing the wood-based feedstock may comprise purchasing the wood-based feedstock. The purchased wood-based feedstock may comprise purchased wood chips or sawdust that originate from wood-based raw material.

Pretreatment in step i) of the wood-based feedstock may comprise one or more different pretreatment steps. During the different pretreatment steps the wood-based feedstock as such changes. The aim of the pretreatment step(s) is to form a slurry for further processing.

The pretreatment i) may comprise subjecting the wood-based feedstock to pre-steaming. The pretreatment i) may comprise subjecting the wood-based feedstock received from the mechanical treatment to pre-steaming. Pretreatment in i) may comprise, before subjecting to the impregnation treatment, subjecting the wood-based feedstock to pre-steaming to form pre-steamed wood-based feedstock. The pretreatment in i) may comprise, an impregnation treatment and a steam explosion treatment and comprise, before subjecting the wood-based feedstock to impregnation treatment and thereafter to steam explosion treatment, subjecting the wood-based feedstock to pre-steaming. The pre-steaming of the wood-based feedstock may be carried out with steam having a temperature of 100-130° C. at atmospheric pressure. During the pre-steaming the wood-based feedstock is treated with steam of low pressure. The pre-steaming may be also carried out with steam having a temperature of below 100° C., or below 98° C., or below 95° C. The pre-steaming has the added utility of reducing or removing air from inside of the wood-based feedstock. The pre-steaming may take place in at least one pre-steaming reactor.

Further, step i) of pretreatment may comprise subjecting the wood-based feedstock to at least one impregnation treatment to form an impregnated wood-based feedstock. Step i) of pretreatment may comprise subjecting the wood-based feedstock to at least one impregnation treatment with an impregnation liquid. The impregnation treatment may be carried out to the wood-based feedstock received from the mechanical treatment and/or from the pre-steaming. The impregnation liquid may be selected from water, at least one acid, at least one alkali, at least one alcohol, or any combination or mixture thereof.

The wood-based feedstock may be transferred from the mechanical treatment and/or from the pre-steaming to the impregnation treatment with a feeder. The feeder may be a screw feeder, such as a plug screw feeder. The feeder may compress the wood-based feedstock during the transfer. When the wood-based feedstock is then entering the impregnation treatment, it may become expanded and absorbs the impregnation liquid.

The impregnation liquid may comprise water, at least one acid, at least one alkali, at least one alcohol, or any combination or mixture thereof. The at least one acid may be selected from a group consisting of inorganic acids, such as sulphuric acid (H₂SO₄), nitric acid, phosphoric acid; organic acids, such as acetic acid, lactic acid, formic acid, carbonic acid; and any combination or mixture thereof. In one embodiment, the impregnation liquid comprises sulphuric acid, e.g. dilute sulphuric acid. The concentration of the acid may be 0.3-5.0% w/w, 0.5-3.0% w/w, 0.6-2.5% w/w, 0.7-1.9% w/w, or 1.0-1.6% w/w. The impregnation liquid may act as a catalyst in affecting the hydrolysis of the hemicellulose in the wood-based feedstock. In one embodiment, the impregnation is conducted by using only water, i.e. by autohydrolysis. In one embodiment, the wood-based feedstock may be impregnated through alkaline hydrolysis. NaOH and Ca₂(OH)₃ can be mentioned as examples to be used as the alkali in the alkaline hydrolysis.

The impregnation treatment may be conducted in at least one impregnation reactor or vessel. In one embodiment, two or more impregnation reactors are used. The transfer from one impregnation reactor to another impregnation reactor may be carried out with a screw feeder.

The impregnation treatment may be carried out by conveying the wood-based feedstock through at least one impregnation reactor that is at least partly filled with the impregnation liquid, i.e. the wood-based feedstock may be transferred into the impregnation reactor, where it sinks into the impregnation liquid, and transferred out of the impregnation reactor such that the wood-based feedstock is homogenously impregnated with the impregnation liquid. As a result of the impregnation treatment, impregnated wood-based feedstock is formed. The impregnation treatment may be carried out as a batch process or in a continuous manner.

The residence time of the wood-based feedstock in an impregnation reactor, i.e. the time during which the wood-based feedstock is in contact with the impregnation liquid, may be 5 seconds-5 minutes, or 0.5-3 minutes or about 1 minute. The temperature of the impregnation liquid may be e.g. 20-99° C., or 40-95° C., or 60-93° C. Keeping the temperature of the impregnation liquid below 100° C. has the added utility of hindering or reducing hemicellulose from dissolving.

After the impregnation treatment, the impregnated wood-based feedstock may be allowed to stay in e.g. a storage tank or a silo for a predetermined period of time to allow the impregnation liquid absorbed into the wood-based feedstock to stabilize. This predetermined period of time may be 15-60 minutes, or e.g. about 30 minutes.

In one embodiment, the wood-based feedstock is subjected to an impregnation treatment with dilute sulphuric acid having a concentration of 1.32% w/w and a temperature of 92° C.

Pretreatment i) may comprise subjecting the wood-based feedstock to steam explosion treatment. The wood-based feedstock from the impregnation treatment may be subjected to steam explosion treatment. I.e. pretreatment i) may comprise subjecting the impregnated wood-based feedstock to steam explosion treatment to form a steam-treated wood-based feedstock.

In one embodiment, pretreatment in i) comprises mechanical treatment of wood-based material to form a wood-based feedstock, the pre-steaming of the wood-based feedstock to form pre-steamed feedstock, impregnation treatment of the pre-steamed wood-based feedstock to form impregnated wood-based feedstock, and the steam explosion treatment of the impregnated wood-based feedstock. In one embodiment, pretreatment in i) comprises pre-steaming the wood-based feedstock, impregnation treatment of the pre-steamed wood-based feedstock, and steam explosion treatment of the impregnated wood-based feedstock. In one embodiment, pretreatment in i) comprises impregnation treatment of the wood-based feedstock, and steam explosion treatment of the impregnated wood-based feedstock. I.e. the wood-based feedstock having been subjected to the impregnation treatment may thereafter be subjected to the steam explosion treatment. Also, the wood-based feedstock having been subjected to pre-steaming, may then be subjected to the impregnation treatment and thereafter the impregnated wood-based feedstock having been subjected to the impregnation treatment may be subjected to steam explosion treatment.

The wood-based feedstock can be stored in e.g. chip bins or silos between the different treatments. Alternatively, the wood-based feedstock may be conveyed from one treatment to the other in a continuous manner.

The pretreatment in i) may comprise subjecting the impregnated wood-based feedstock to steam explosion treatment that is carried out by treating the impregnated wood-based feedstock with steam having a temperature of 130-240° C. under a pressure of 0.17-3.25 MPaG followed by a sudden, explosive decompression of the feedstock. The feedstock may be treated with the steam for 1-20 minutes, or 1-20 minutes, or 2-16 minutes, or 4-13 minutes, or 3-10 minutes, or 3-8 minutes, before the sudden, explosive decompression of the steam-treated wood-based feedstock.

In this specification, the term “steam explosion treatment” may refer to a process of hemihydrolysis in which the feedstock is treated in a reactor (steam explosion reactor) with steam having a temperature of 130-240° C. under a pressure of 0.17-3.25 MPaG followed by a sudden, explosive decompression of the feedstock that results in the rupture of the fiber structure of the feedstock.

In one embodiment, the amount of sulphuric acid in the steam explosion treatment may be 0.10-0.75 weight-% based on the total dry matter content of the wood-based feedstock. The amount of acid present in the steam explosion treatment may be determined by measuring the sulphur content of the liquid of the steam-treated wood-based feedstock or the liquid part of the steam-treated wood-based feedstock after steam explosion treatment. The amount of sulphuric acid in the steam explosion reactor may be determined by subtracting the amount of sulphur in the wood-based feedstock from the measured amount of total sulphur in the steam-treated wood-based feedstock.

The steam explosion treatment may be conducted in a pressurized reactor. The steam explosion treatment may be carried out in the pressurized reactor by treating the impregnated wood-based feedstock with steam having a temperature of 130-240° C. under a pressure of 0.17-3.25 MPaG followed by a sudden, explosive decompression of the feedstock. The impregnated wood-based feedstock may be introduced into the pressurized reactor with a compressing conveyor, e.g. a screw feeder. During transportation with the screw feeder, if used, the acid in liquid form is remover, and part of the impregnation liquid absorbed by the feedstock is removed as a pressate while most of it remains in the feedstock. The impregnated wood-based feedstock may be introduced into the pressurized reactor along with steam and/or gas. The pressure of the pressurized reactor can be controlled by the addition of steam. The pressurized reactor may operate in a continuous manner or as a batch process. The impregnated wood-based feedstock, e.g. the wood-based feedstock that has been subjected to an impregnation treatment, may be introduced into the pressurized reactor at a temperature of 25-140° C. The residence time of the feedstock in the pressurized reactor may be 0.5-120 minutes. The term “residence time” should in this specification, unless otherwise stated, be understood as the time between the feedstock being introduced into or entering e.g. the pressurized reactor and the feedstock being exited or discharged from the same.

As a result of the hemihydrolysis of the wood-based feedstock affected by the steam explosion treatment in the reactor, the hemicellulose present in the wood-based feedstock may become hydrolyzed or degraded into e.g. xylose oligomers and/or monomers. The hemicellulose comprises polysaccharides such as xylan, mannan and glucan. Xylan is thus hydrolyzed into xylose that is a monosaccharide. In one embodiment, the conversion of xylan present in the wood-based feedstock into xylose as a result of the hemihydrolysis is 87-95%, or 83-93% or 90-92%.

Thus, steam explosion of the feedstock may result in the formation of a steam-treated wood-based feedstock. The steam-treated wood-based feedstock from the steam explosion may be subjected to steam separation. The steam-treated wood-based feedstock from the steam explosion may be mixed or combined with a liquid, e.g. water. The steam-treated wood-based feedstock from the steam explosion treatment may be mixed with a liquid to form a slurry. The liquid may be pure water or water containing C5 sugars. The water containing C5 sugars may be recycled water from separation and/or washing the fraction comprising solid cellulose particles before enzymatic hydrolysis. The steam-treated wood-based feedstock may be mixed with the liquid and the resulting mass may be homogenized mechanically to break up agglomerates. Pretreatment in i) may comprise mixing the steam-treated wood-based feedstock with a liquid.

As a result of the pretreatment i) a slurry may thus be formed. The slurry may comprise a liquid phase and a solid phase. The slurry may comprise solid cellulose particles. In step ii) the slurry may be separated into a liquid fraction and a fraction comprising solid cellulose particles.

The method comprises ii) of separating a liquid fraction and a fraction comprising solid cellulose particles by a first solid-liquid separation process, wherein the first solid-liquid separation process comprises washing. In one embodiment, washing in step ii) is continued until the amount of soluble organic components in the fraction comprising solid cellulose particles is 0.5-5 weight-%, or 1-4 weight-%, or 1.5-3 weight-% based on the total dry matter content. In one embodiment, washing in step ii) is continued until the amount of soluble organic components in the fraction comprising solid cellulose particles is 0.5-5 weight-%, or 1-4 weight-%, or 1.5-3 weight-% based on the total dry matter content of the fraction comprising solid cellulose particles. In one embodiment, a fraction comprising solid cellulose particles having a total dry matter content of 15-50 weight-%, or 21-40 weight-%, or 25-40 weight-%, or 30-40 weight-%, or 35-40 weight-% is formed in ii).

In one embodiment, the first solid-liquid separation process in step ii) is carried out by displacement washing or countercurrent washing. Thus, the first solid-liquid separation process may be selected from displacement washing and countercurrent washing.

Displacement washing, or replacement washing as it may also be called, is a method for separating solids and liquid from each other by the use of a rather minor amount of washing liquid. Thus, displacement washing may be considered as an operation by which it is possible to wash solid particles with a minimum amount of washing liquid, such as water.

In countercurrent washing, the movement of the fraction comprising solid cellulose particles in generally in a forward direction, whereas the washing liquid, such as water, flows in the opposite direction. As for the displacement washing, also the countercurrent washing may reduce the consumption of washing liquid to a great extent.

In one embodiment, countercurrent washing comprises at least two solid-liquid separation steps and one dilution in between the steps with washing solution. The washing solution may be clean water. The amount of water needed may vary depending on how many solid-liquid separation steps are performed in total, the total dry matter content in the feed of the solid-liquid separation step and the total dry matter content in the fraction comprising solid cellulose particles after each solid-liquid separation step.

The washing liquid may be fresh washing water or recycled washing water. The washing water may be fresh water, drinking water, or a sugar containing liquid with low sugar content. The conductivity of the washing liquid may be about 0.1 ms/cm.

The ratio of the used washing liquid to the solids in step ii) may be 0.5:1-8:1 (w/w), or 0.5:1-5:1 (w/w), or 0.5:1-3:1 (w/w), or 0.5:1-2:1 (w/w) in the case of displacement washing.

The progression of the displacement washing as well as of the countercurrent washing may be monitored by measuring the conductivity of the liquid fraction recovered from this treatment. Once the conductivity of the liquid fraction is below or equal to a predetermined threshold value of 0.35 mS/cm, one may conclude that that the desired amount of the C5 sugars and other soluble impurities have been removed and the washing may be concluded. In one embodiment, the washing is continued until the conductivity of the liquid fraction is 0.1-1.0 mS/cm or 0.2-0.5 mS/cm.

As a result of step ii) a fraction comprising solid cellulose particles having a total dry matter content of 15-50 weight-% is formed.

The inventors surprisingly found out that by separating the liquid fraction and the fraction comprising solid cellulose particles from each other by the first solid-liquid separation process, e.g. by displacement washing or countercurrent washing, beneficially reduced the amount of C5 sugars from the fraction comprising solid cellulose particles, thereby affecting the outcome of the method, i.e. the properties of the carbohydrate composition, to a rather great extent. The method as disclosed in the current specification has the added utility of resulting in a carbohydrate composition of high quality or purity in view of the same being used in further applications.

The separated liquid fraction may thus comprise C5 sugars from hydrolyzed hemicellulose as well as solutile lignin and other by-products.

The fraction comprising solid cellulose particles may, in addition to cellulose, comprise lignin. As the C5 sugars are efficiently removed with the liquid fraction, the fraction comprising solid cellulose particles may comprise carbohydrates such as solid C6 sugars. The fraction comprising solid cellulose particles may also comprise other carbohydrates and other components. The fraction comprising solid cellulose particles may also comprise some amount of C5 sugars.

The separated and recovered fraction comprising solid cellulose particles may be further purified or washed before being subjected to enzymatic hydrolysis.

In one embodiment, the separated fraction comprising solid cellulose particles is diluted in iii) to a total dry matter content of 8-20 weight-%, or 10-18 weight-%, or 15-16 weight-%. Thus, if needed, the separated fraction comprising solid cellulose particles is diluted in step iii). The need to dilute is dependent on the total dry matter content that the fraction comprising solid cellulose particles may have as a result of step ii). I.e. if the total dry matter content of the fraction comprising solid cellulose particles as a result of step ii) is higher than 20 weight-%, then the fraction comprising solid cellulose particles may be diluted. If the total dry matter content of the fraction comprising solid cellulose particles as a result of step ii) is 8-20 weight-%, then no dilution may be needed. The fraction comprising solid cellulose particles may be diluted with water and/or other liquid containing at least soluble carbohydrates. In one embodiment, the fraction comprising solid cellulose particles may be diluted in step iii) with water to a total dry matter content of 8-20 weight-%, or 10-18 weight-%, or 15-16 weight-%.

In one embodiment, the separated fraction comprising solid cellulose particles is subjected to enzymatic hydrolysis to form a hydrolysis product, wherein the fraction comprising solid cellulose particles has a total dry matter content of 8-20 weight-% when being subjected to enzymatic hydrolysis.

Step iv) of subjecting the fraction comprising solid cellulose particles to enzymatic hydrolysis may be carried out at a temperature of 30-70° C., or 35-65° C., or 40-60° C., or 42-59° C., or 45-58° C., or 47-57° C. Step iv) of subjecting the fraction comprising solid cellulose particles to enzymatic hydrolysis may be carried out at atmospheric pressure. The pH of the fraction comprising solid cellulose particles may be kept during iv) at a pH value of 3.5-6.5, or 4.0-6.0, or 4.5-5.5. The pH of the fraction comprising solid cellulose particles can be adjusted with the addition of alkali and/or acid. iv) of subjecting the fraction comprising solid cellulose particles to enzymatic hydrolysis may be continued for 20-120 h, or 30-90 h, or 40-80 h. The enzymatic hydrolysis of the fraction comprising solid cellulose particles may be carried out in a continuous manner or as a batch-type process or as a combination of a continuous and a batch-type process.

In one embodiment, the enzymatic hydrolysis is carried out at a temperature of 30-70° C., or 35-65° C., or 40-60° C., or 45-55° C., or 48-53° C. while keeping the pH of the fraction comprising solid cellulose particles at a pH value of 3.5-6.5, or 4.0-6.0, or 4.5-5.5, and wherein the enzymatic hydrolysis is allowed to continue for 20-120 h, or 30-90 h, or 40-80 h.

The enzymatic hydrolysis may be conducted in at least one process step.

In one embodiment, the enzymatic hydrolysis may be carried out as a one-step hydrolysis process, wherein the fraction comprising solid cellulose particles is subjected to enzymatic hydrolysis in at least one first hydrolysis reactor. After the hydrolysis, the hydrolysis product, i.e. the hydrolysate, may be subjected to a separation, wherein the solid fraction comprising lignin, which in addition to lignin may also comprise non-hydrolyzed cellulose, is separated from the liquid carbohydrate fraction. The one-step hydrolysis process may be carried out as a batch process comprising e.g. several reactors working in parallel, wherein each reactor may receive a part of the fraction comprising solid cellulose particles. Further, separate parallel lines with parallel reactors may be used.

In one embodiment, the enzymatic hydrolysis may be carried out as a two-step hydrolysis process or as a multi-step hydrolysis process. In the two-step hydrolysis process or in the multi-step hydrolysis process the fraction comprising solid cellulose particles may first be subjected to a first enzymatic hydrolysis in at least one first hydrolysis reactor. Then the formed liquid carbohydrate fraction may be separated from the solid fraction comprising lignin, which may also comprise unhydrolyzed cellulose. The solid fraction may then be subjected to a second or any latter enzymatic hydrolysis, e.g. in at least one second hydrolysis reactor. At least one of the first enzymatic hydrolysis and the second or any latter enzymatic hydrolysis may be carried out as a batch process or as a continuous process comprising e.g. one or several reactors working in parallel. After the second or any latter enzymatic hydrolysis, the hydrolysis product, i.e. the hydrolysate, may be subjected to separation, wherein the solid fraction comprising lignin is separated from the liquid carbohydrate fraction.

The reaction time in the first hydrolysis reactor may be 8-72 hours. The reaction time in the second and/or any latter hydrolysis reactor may be 8-72 hours.

The enzymes are catalysts for the enzymatic hydrolysis. The enzymatic reaction decreases the pH and by shortening the length of the cellulose fibers it may also decrease the viscosity. Subjecting the fraction comprising solid cellulose particles to enzymatic hydrolysis may result in cellulose being transformed into glucose monomers with enzymes. Lignin present in the fraction comprising solid cellulose particles may remain essentially in solid form.

At least one enzyme may be used for carrying out the enzymatic hydrolysis. The at least one enzyme may be selected from a group consisting of cellulases, hemicellulases, laccases, and lignolytic peroxidases. Cellulases are multi-protein complexes consisting of synergistic enzymes with different specific activities that can be divided into exo- and endo-cellulases (glucanase) and β-glucosidase (cellobiose). The enzymes may be either commercially available cellulase mixes or on-site manufactured.

Cellulose is an insoluble linear polymer of repeating glucose units linked by β-1-4-glucosidic bonds. During the enzymatic hydrolysis, cellulose chains are broken by means of breaking at least one β-1-4-glucosidic bond.

Enzymatic hydrolysis may result in the formation of hydrolysis product. In step v) the hydrolysis product may be separated into a solid fraction comprising lignin and a liquid carbohydrate fraction by a second solid-liquid separation process to recover the liquid carbohydrate fraction as a wood-derived carbohydrate composition.

During the separation in v) the solid fraction may be separated from the liquid fraction. In one embodiment, step v) comprises separating the solid fraction comprising lignin and the liquid carbohydrate fraction by a second solid-liquid separation process. The separation in step v) may be carried out by filtration, decanting, and/or by centrifugal treatment. The filtration may be vacuum filtration, filtration based on the use of reduced pressure, filtration based on the use of overpressure, or filter pressing. The decanting may be repeated in order to improve separation.

The liquid carbohydrate fraction recovered from enzymatic hydrolysis may be purified after step v). The purification of the liquid carbohydrate fraction may be carried out by using at least one of the following: (membrane) filtration, crystallization, sterilization, pasteurization, evaporation, chromatography, ion exchanging, flocculation, flotation, precipitation, centrifugal separation, microfiltration, ultrafiltration, nanofiltration, osmosis, electrodialysis, thermal treatment, by activated carbon treatment, or by any combination thereof. Purification of the liquid carbohydrate fraction has the added utility of providing a desired target quality of sugars.

The method as disclosed in the current specification has the added utility of providing a wood-derived carbohydrate composition with a high content of monomeric C6 sugars. The wood-derived carbohydrate composition has the added utility of fulfilling purity properties required for further use in e.g. a process of catalytic conversion for the production of e.g. mono-ethylene glycol.

### EXAMPLES

Reference will now be made in detail to the embodiments of the present disclosure, an example of which is illustrated in the accompanying drawing.

The description below discloses some embodiments in such a detail that a person skilled in the art is able to utilize the method based on the disclosure. Not all steps of the embodiments are discussed in detail, as many of the steps will be obvious for the person skilled in the art based on this disclosure.

For reasons of simplicity, item numbers will be maintained in the following exemplary embodiments in the case of repeating components.

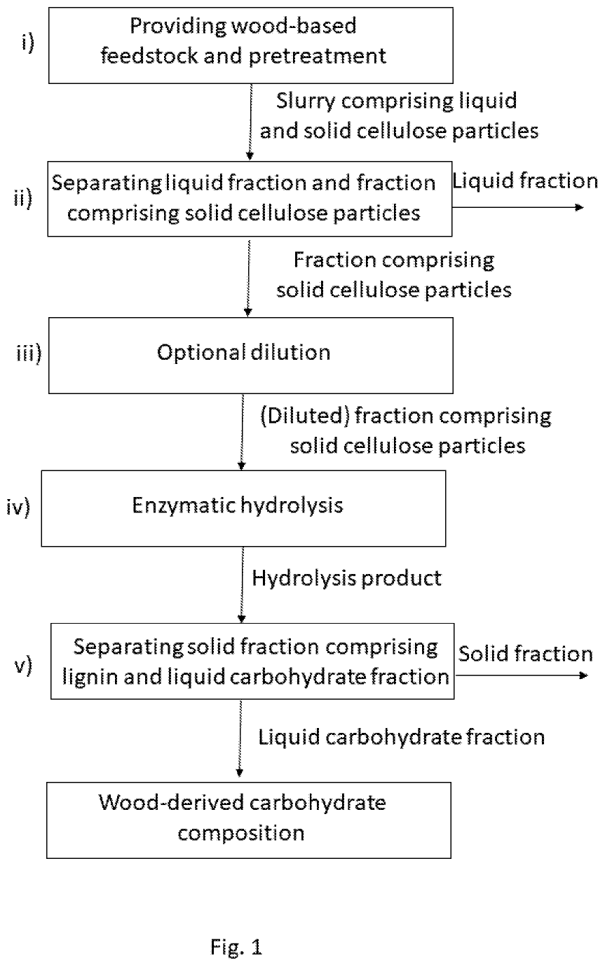

The enclosed FIG. 1 illustrates an embodiment of a flow chart of the method for producing a wood-derived carbohydrate composition in some detail. The method of FIG. 1 for producing a wood-derived carbohydrate composition comprises providing a wood-based feedstock originating from wood-based raw material and comprising wood chips and subjecting the wood-based feedstock to pretreatment to form a slurry (step i) of FIG. 1). The slurry is then separated into a liquid fraction and a fraction comprising solid cellulose particles by a first solid-liquid separation process comprising washing (step ii) of FIG. 1).

The separated fraction comprising solid cellulose particles is then optionally diluted (step iii) of FIG. 1).

Then the fraction comprising solid cellulose particles is subjected to enzymatic hydrolysis to form a hydrolysis product (step iv) of FIG. 1). The hydrolysis product is then separated to form a solid fraction comprising lignin and a liquid carbohydrate fraction by a second solid-liquid separation process to recover the liquid carbohydrate fraction as a wood-derived carbohydrate composition (step v) of FIG. 1).

### Example 1—Producing Wood-Derived Carbohydrate Composition

In this example a wood-derived carbohydrate composition was prepared.

First a wood-based feedstock comprising chips of beech wood was provided. The wood-based feedstock was then subjected to pretreatment in the following manner:

The wood-based feedstock was subjected to pre-steaming. Pre-steaming of the wood-based feedstock was carried out at atmospheric pressure with steam having a temperature of 100° C. for 180 minutes. The pre-steamed feedstock was then subjected to an impregnation treatment with dilute sulphuric acid having a concentration of 1.32% w/w and a temperature of 92° C. The pre-steamed wood-based feedstock was allowed to be affected by the impregnation liquid for 30 minutes. The impregnated wood-based feedstock was then subjected to steam explosion treatment. The steam explosion treatment was carried out by treating the impregnated wood-based feedstock with steam having a temperature of 191° C. at atmospheric pressure followed by a sudden, explosive decompression of the wood-based feedstock. The amount of sulphuric acid in steam explosion reactor was 0.33 weight-% based on the total dry matter content of the wood-based feedstock. In the determination of the amount of sulphuric acid the sulphur content of wood was 0.02 weight-% based on the total dry matter content of the wood used.

In the pretreatment, the conversion of xylan in the wood-based feedstock into xylose was 91% and the ratio of solubilized glucose to solubilized xylose was approximately 0.15 as determined by HPLC-RI. The steam-treated wood-based feedstock was then mixed with water in a mixing vessel.

As a result of the above pretreatment steps, a slurry was formed. The slurry comprised a liquid fraction and a fraction comprising solid cellulose particles. The fraction comprising solid cellulose particles also comprised lignin. The slurry was then separated into the liquid fraction and the fraction comprising solid cellulose particles by a first solid-liquid separation process, which in this example was countercurrent washing. The countercurrent washing was continued until the amount of soluble components in the fraction comprising solid cellulose particles was 2.0 weight-% based on the total dry matter content. The total dry matter content of the fraction comprising solid cellulose particles was 32 weight-% after the washing.

The resulting fraction comprising solid cellulose particles with the total dry matter content of 32 weight-% was diluted to a total dry matter content of approximately 13 weight-%, and was then subjected to enzymatic hydrolysis in a batch reactor by using the following conditions:

initial pH=5.0 adjusted by NaOH enzyme=Commercially available cellulase mixture

residence time=53 hours

temperature=47-52° C. during the process

The dosing of the cellulase mixture was selected such that the conversion of glucose after 53 hours was 83%.

The enzymatic hydrolysis resulted in a hydrolysis product. The hydrolysis product was then separated into a solid fraction comprising lignin and a liquid carbohydrate fraction. These were separated from each other by using a decanter centrifuge in a two-step washing process. The carbohydrate concentration of the liquid carbohydrate fraction in the first washing step was approximately 8 weight-% and in the second washing step approximately 4 weight-% after reslurrying.

The liquid carbohydrate fraction was recovered as a wood-derived carbohydrate composition that was analyzed by HPLC-RI using a Waters e2695 Alliance Separation module, a Waters 2998 Photodiode Array, and a Waters 2414 Refractive Index detector. Separation was achieved with a Bio-Rad Aminex HPX-87 column with dimensions 300 mm×7.8 mm equipped with Micro-Guard Deashing and Carbo-P guard columns in series. Ultrapure water was used as eluent. The results are presented in the below table:

| | Weight-% based on the total |
|---|---|
| | dry matter content |
| Monomeric sugars | |
| Glucose, HPLC-RI | 83.5% |
| Xylose, HPLC-RI | 5.8% |
| Galactose, HPLC-RI | 0.2% |
| Arabinose, HPLC-RI | 0.1% |
| Mannose, HPLC-RI | 0.1% |
| Fructose, HPLC-RI | 0.0% |
| Oligomeric sugars | |
| Glucose, acid | 1.3% |
| hydrolysis, HPLC-RI | |
| Xylose, acid | 0.0% |
| hydrolysis, HPLC-RI | |
| Galactose, acid | 0.4% |
| hydrolysis, HPLC-RI | |
| Arabinose, acid | 0.0% |
| hydrolysis, HPLC-RI | |
| Mannose, acid | 0.7% |
| hydrolysis, HPLC-RI | |
| Fructose, acid | 0.0% |
| hydrolysis, HPLC-RI | |
| Olig. carbohydrates | 2.4% |
| total, acid | |
| hydrolysis, HPLC-RI | |
| Ash 525° C. | 0.90% |
| Lignin, soluble, UV | 1.66% |
| 205 | |
| Carboxylic acids, | 1.76% |
| total, HPLC-PDA | |
| Sulphur, S, ICP | 0.04% |

The amount of oligomeric sugars in the sample was determined by hydrolyzing the oligomeric sugars into monomeric sugars using acid hydrolysis, analyzing the acid hydrolyzed sample using HPLC-RI, and comparing the result to those for samples for which the hydrolysis was not performed. By subtracting the amount of monomeric sugars in the untreated sample, the amount of oligomeric sugars was calculated.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A wood-derived carbohydrate composition or a method disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A wood-derived carbohydrate composition comprising monomeric C6 sugars and monomeric C5 sugars in a total amount of at least 80 weight-% based on the total dry matter content of the carbohydrate composition, wherein the ratio of the monomeric C5 sugars to the monomeric C6 sugars is at most 0.15.

2. The wood-derived carbohydrate composition of claim 1, wherein the carbohydrate composition comprises monomeric C6 sugars and monomeric C5 sugars in a total amount of 80-95 weight-%, based on the total dry matter content of the carbohydrate composition.

3. The wood-derived carbohydrate composition of claim 1, wherein the carbohydrate composition comprises oligomeric C6 sugars and oligomeric C5 sugars in a total amount of 0.5-5 weight-%, based on the total dry matter content of the carbohydrate composition.

4. The wood-derived carbohydrate composition of claim 1, wherein the ratio of monomeric C5 sugars to the monomeric C6 sugars in the carbohydrate composition is 0.015-0.15.

5. The wood-derived carbohydrate composition of claim 1, wherein the carbohydrate composition comprises soluble lignin in an amount of at most 5 weight-%, based on the total dry matter content of the carbohydrate composition.

6. The wood-derived carbohydrate composition of claim 1, wherein the carbohydrate composition comprises organic impurities, inorganic impurities, or a combination thereof in an amount of at most 20 weight-%, based on the total dry matter content of the carbohydrate composition.

7. The wood-derived carbohydrate composition of claim 1, wherein the composition comprises sulphur in an amount of 0.01-0.3 weight-%, based on the total dry matter content of the carbohydrate composition.

8. The wood-derived carbohydrate composition of claim 1, wherein the carbohydrate composition comprises nitrogen in an amount of 0.01-1.0 weight-%, based on the total dry matter content of the carbohydrate composition when measured as total nitrogen content of the carbohydrate composition.

9. The wood-derived carbohydrate composition of claim 1, wherein the monomeric C6 sugars are present in an amount of at least 80 weight-%, based on the total dry matter content of the carbohydrate composition.

10. The wood-derived carbohydrate composition of claim 1, wherein the monomeric C5 sugars are present in an amount of at most 10 weight-%, based on the total dry matter content of the carbohydrate composition.

11. The wood-derived carbohydrate composition of claim 1, wherein the conductivity of a 10% aqueous solution of the carbohydrate composition is 0.5-10 mS/cm, when determined according to SFS-EN 27888.

12. The wood-derived carbohydrate composition of claim 1, wherein the ICUMSA color value of an aqueous solution of the carbohydrate composition is 10 000-50 000 IU.

13. A method for producing a wood-derived carbohydrate composition, wherein the method comprises:
i) providing a wood-based feedstock originating from wood-based raw material and comprising wood chips, and subjecting the wood-based feedstock to pretreatment to form a slurry;
ii) separating the slurry into a liquid fraction and a fraction comprising solid cellulose particles by a first solid-liquid separation process to form a fraction comprising solid cellulose particles having a total dry matter content of 15-50 weight-%, wherein the first solid-liquid separation process comprises washing the fraction comprising solid cellulose particles until the amount of soluble organic components in the fraction comprising solid cellulose particles is 0.5-5 weight-% based on the total dry matter content;
iii) optionally, diluting the separated fraction comprising solid cellulose particles to a total dry matter content of 8-20 weight-%;
iv) subjecting the fraction comprising solid cellulose particles to enzymatic hydrolysis to form a hydrolysis product, wherein the fraction comprising solid cellulose particles has a total dry matter content of 8-20 weight-%; and
v) separating the hydrolysis product into a solid fraction comprising lignin and a liquid carbohydrate fraction by a second solid-liquid separation process to recover the liquid carbohydrate fraction as a wood-derived carbohydrate composition.

14. The method of claim 13, wherein pretreatment in i) comprises subjecting the wood-based feedstock to at least one impregnation treatment to form an impregnated wood-based feedstock.

15. The method of claim 14, wherein pretreatment in i) comprises subjecting the impregnated wood-based feedstock to steam explosion treatment to form a steam-treated wood-based feedstock.

16. The method of claim 15, wherein pretreatment in i) comprises mixing the steam-treated wood-based feedstock with a liquid.

17. The method of claim 13, wherein pretreatment in i) comprises, before subjecting to the impregnation treatment, subjecting the wood-based feedstock to pre-steaming to form pre-steamed wood-based feedstock.

18. The method of claim 13, wherein first solid-liquid separation process in ii) is carried out by displacement washing or countercurrent washing.

19. The method of claim 13, wherein washing in ii) is continued until the amount of soluble organic components in the fraction comprising solid cellulose particles is 1-4 weight-%, based on the total dry matter content.

20. The method of claim 13, wherein a fraction comprising solid cellulose particles having a total dry matter content of 21-40 weight-% is formed in ii).

21. The method of claim 13, wherein the separated fraction comprising solid cellulose particles is diluted in iii) to a total dry matter content of 10-18 weight-%.

22. The method of claim 13, wherein the enzymatic hydrolysis is carried out at a temperature of 30-70° C., while keeping the pH of the fraction comprising solid cellulose particles at a pH value of 3.5-6.5, and wherein the enzymatic hydrolysis is allowed to continue for 20-120 h.

23. (canceled)

24. (canceled)
